(19) United States
(12) Patent Application Publication
(10) Pub. No.: US 2026/0245729 A1
(43) Pub. Date: Aug. 20, 2026

(54) ELECTRONIC DEVICE, AND CONTROL METHOD AND CONTROL PROGRAM FOR ELECTRONIC DEVICE

(71) Applicant: KYOCERA Corporation, Kyoto-shi, Kyoto (JP)

(72) Inventors: Masaya KOBAYASHI, Kawasaki-shi, Kanagawa (JP); Jungo MIYAZAKI, Kawasaki-shi, Kanagawa (JP)

(73) Assignee: KYOCERA Corporation, Kyoto-shi, Kyoto (JP)

(21) Appl. No.: 18/871,386

(22) PCT Filed: Jun. 5, 2023
(86) PCT No.: PCT/JP2023/020856

§ 371 Date: Dec. 3, 2024

(30) Foreign Application Priority Data
Jun. 15, 2022 (JP) 2022-096809

(51) Int. Cl. G16H 50/30; A61B 5/16

(52) U.S. Cl. CPC G16H 50/30; A61B 5/165

(57) ABSTRACT

An electronic device includes a controller configured to estimate an internal state of a subject in accordance with a measurement result of biological information on the subject in each of a first period and a second period later than the first period. The controller is configured to: generate proforma information corresponding to a proforma internal state of the subject in the first period; predict the biological information on the subject in the second period in accordance with the proforma internal state of the subject and the measurement result of the biological information on the subject in the first period; and estimate an internal state corresponding to the biological information in accordance with a difference between the predicted biological information and the measurement result of the biological information on the subject in the second period.

### CROSS-REFERENCE TO RELATED APPLICATIONS

This application claims priority from Japanese Patent Application No. 2022-96809 (filed Jun. 15, 2022) and the entire content of the application is incorporated herein by reference.

### TECHNICAL FIELD

The disclosure relates to an electronic device, and a control method and a control program for the electronic device.

### BACKGROUND OF INVENTION

Study on estimation of an internal state, such as a concentration level or emotion of a subject, has been recently underway. For example, in a reported test, a state of mind of a learner is estimated by recording during a lecture what a teacher has said, biological information on the learner and a video of the learner and then by introspective reporting performed by the learner about emotion of the learner themselves at each scene after the lecture (see Non Patent Literature 1).

### CITATION LIST

### Non Patent Literature

Non Patent Literature 1: Tatsunori Matsui, Tatsuro Uno, Yoshimasa Tawatsuji “Study on Estimation of Learner's Mental States from Physiological Indexes Considering Time Dilation and Persistent Model of Mental States”, Year 2018 Annual Conference of the Japanese Society for Artificial Intelligence (32nd), General Incorporated Association, Society for Artificial Intelligence

### SUMMARY

In an embodiment of the disclosure, an electronic device includes a controller configured to estimate an internal state of a subject in accordance with a measurement result of biological information on the subject in each of a first period and a second period later than the first period. The controller is configured to generate proforma information corresponding to a proforma internal state of the subject in the first period. The controller is configured to predict the biological information on the subject in the second period in accordance with the proforma internal state of the subject and the measurement result of the biological information on the subject in the first period. The controller is configured to estimate an internal state corresponding to the biological information in accordance with a difference between the predicted biological information and the measurement result of the biological information on the subject in the second period.

In an embodiment of the disclosure, a control method for an electronic device is executed at the electronic device that estimates an internal state of a subject in accordance with a measurement result of biological information on the subject in each of a first period and a second period later than the first period. In the control method for the electronic device, the electronic device generates proforma information indicating each of a plurality of states assuming the internal state of the subject. In the control method for the electronic device, the electronic device generates proforma information corresponding to a proforma internal state of the subject in the first period. In the control method for the electronic device, the electronic device predicts the biological information on the subject in the second period in accordance with the proforma internal state of the subject and the measurement result of the biological information on the subject in the first period. In the control method for the electronic device, the electronic device estimates an internal state corresponding to the biological information in accordance with a difference between the predicted biological information and the measurement result of the biological information on the subject in the second period.

In an embodiment of the disclosure, a control program for an electronic device is executed at the electronic device estimating an internal state of a subject in accordance with a measurement result of biological information on the subject in each of a first period and a second period later than the first period. The control program for the electronic device causes the electronic device to generate proforma information indicating each of a plurality of states assuming the internal state of the subject. The control program for the electronic device causes the electronic device to generate proforma information corresponding to a proforma internal state of the subject in the first period. The control program for the electronic device causes the electronic device to predict the biological information on the subject in the second period in accordance with the proforma internal state of the subject and the measurement result of the biological information on the subject in the first period. The control program for the control method causes the electronic device to estimate an internal state corresponding to the biological information in accordance with a difference between the predicted biological information and the measurement result of the biological information on the subject in the second period.

### BRIEF DESCRIPTION OF THE DRAWINGS

FIG. 1 is a block diagram illustrating a schematic configuration of an electronic device in an embodiment.

FIG. 2 is a conceptual diagram explaining an example of encoding of the electronic device.

FIG. 3 is a conceptual diagram explaining an example of decoding of the electronic device.

FIG. 4 is a conceptual diagram explaining an operation of an autoencoder in the electronic device.

FIG. 5 is a diagram illustrating a configuration example of a prediction model related to an embodiment.

FIG. 6 is a diagram illustrating an example of a signal waveform of biological information on a subject.

FIG. 7 is a flowchart illustrating an operation example of a method of generating a prediction model.

FIG. 8 is a flowchart illustrating an operation example of a method of estimating an internal state.

FIG. 9 is a flowchart illustrating an operation example of a method of estimating an internal state in view of a certainty factor of an estimation result of the internal state.

### DESCRIPTION OF EMBODIMENTS

An internal state of a subject may correlate with biological information on the subject at a given time point. Specifically, the biological information on the subject at the given time point may reflect the internal state of the subject at the given time point. On the other hand, a chronological change in the biological information on the subject may also reflect the internal state of the subject. An estimation accuracy of the internal state may be expected to be improved by factoring in a chronological change of the biological information. Improvement of the estimation accuracy of the internal state of the subject may be in demand.

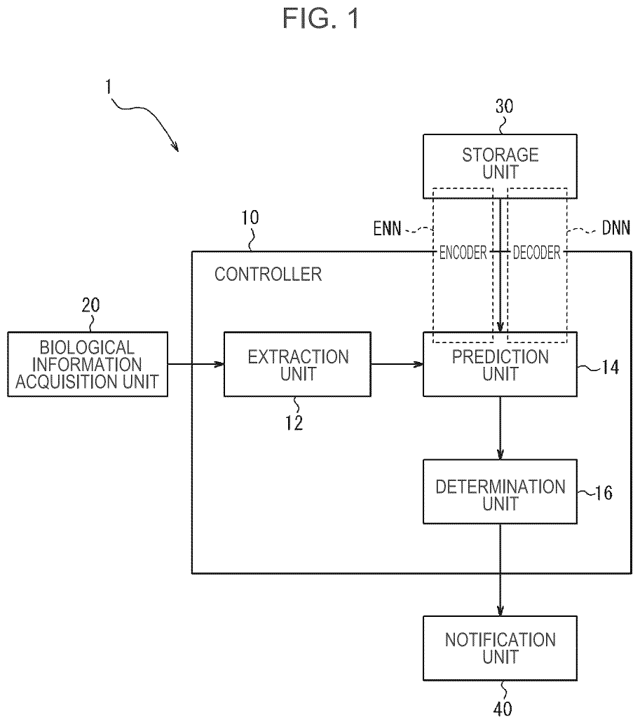

Referring to the drawings, an embodiment of an electronic device 1 of the disclosure (see FIG. 1) is described below. The following description also serves as a description in an embodiment of a control method and a control program for the electronic device 1 of the disclosure.

In the disclosure, “the electronic device 1” may be a device that is electric-power driven. In an embodiment, the electronic device 1 estimates an internal state of a subject, such as emotion or a concentration level of the subject. The “subject” may be an individual serving as a target (typically a human) from which the electronic device 1 in an embodiment estimates an internal state. Also, in the disclosure, a “user” may be an individual (typically a human) that uses the electronic device 1 in an embodiment. The “user” may be the same individual as the “subject”, or may be a different individual. The “user” and the “subject” may be humans or animals other than humans.

In an embodiment, the electronic device 1 may be each of a variety of devices. For example, the electronic device 1 in an embodiment may be a specially designed terminal or any device, such as a general-purpose smart phone, a tablet, a phablet, a laptop (notebook PC), a computer, or a server. For example, as a cellular phone or a smart phone, the electronic device 1 in an embodiment may have a function capable of communicating with another device. “Another device” described herein may be a device, such as a cellular phone or a smart phone, or may be any device, such as a base station, a server, a dedicated terminal, or a computer. In the disclosure, “another device” may be a device, an apparatus, or the like, driven by electric power. In an embodiment, the electronic device 1 may be configured to be wiredly and/or wirelessly communicable with another device.

In an example in an embodiment described below, the electronic device 1 is a server, a device, or the like, connected to an online conference. In this case, the electronic device 1 may estimate a specific internal state of a participant of the online conference (for example, a specific state of mind, emotion, a concentration level, or the like). The electronic device 1 may notify either a conference organizer or a facilitator, or a participant themselves of an estimation result of the internal state of the participant, or may output an alert responsive to the estimation result of the internal state of the participant.

### (Configuration Example of Electronic Device 1)

Referring to FIG. 1, the electronic device 1 in an embodiment may include a controller 10, a biological information acquisition unit 20, a storage unit 30, and a notification unit 40. Referring to FIG. 1, the controller 10 may include an extraction unit 12, a prediction unit 14, and a determination unit 16. The electronic device 1 in an embodiment may include all functional units illustrated in FIG. 1, or may not include at least some of the functional units illustrated in FIG. 1. For example, in an embodiment, the electronic device 1 may include only the controller 10 illustrated in FIG. 1. In such a case, the electronic device 1 in an embodiment may be connected to the biological information acquisition unit 20, the storage unit 30, the notification unit 40 and the like, prepared as external devices. Functions of at least one selected from the group consisting of the controller 10, the prediction unit 14 and the storage unit 30 may realize functions of an encoder ENN and a decoder DNN described below. Input information or input data may be transmitted to, for example, the extraction unit 12, the encoder ENN, the decoder DNN, and the determination unit 16 in this order. The encoder ENN may output a latent variable Z described below. In this case, the decoder DNN may receive the output latent variable Z. In the disclosure, the biological information acquisition unit 20 may include an imaging unit that acquires at least one of a still image or a moving image and an acquisition unit that acquires one or more feature values including a heart rate, a heart sound, a temperature, a line of sight and a movement of the subject.

The controller 10 controls and/or manages the whole electronic device 1 including each functional unit configuring the electronic device 1. In order to provide control and a processing capability used to execute a variety of functions, the controller 10 may include at least one processor, such as a CPU (Central Processing Unit) or a DSP (Digital Signal Processor). A single processor as a whole may implement the controller 10, a plurality of processors may implement the controller 10, or each individual processor may implement the controller 10. A single integrated circuit may implement the processor. The integrated circuit is also referred to as IC (Integrated Circuit). The processor may be implemented as a plurality of integrated circuits and discrete circuits that are communicably connected to each other. The processor may be implemented using a variety of other related art techniques.

The controller 10 may include one or more processors and a memory. The processor may include a general-purpose processor that executes a special function by reading a special program and a dedicated processor that specializes in executing a specific process. The dedicated processor may include an IC (ASIC: Application Specific Integrated Circuit) for specific applications. The processor may include a programmable logic device (PLD: Programmable Logic Device). The PLD may include FPGA (Field-Programmable Gate Array). The controller 10 may be SoC (System-on-a-Chip) in which one or more processors operate in cooperation, or may be SiP (System In a Package). The controller 10 controls operation of each configuration element of the electronic device 1.

The controller 10 may include, for example, at least one of a software resource or a hardware resource. In the electronic device 1 in an embodiment, the controller 10 may include specific means in which a software resource and a hardware resource operate in cooperation. At least one selected from the group consisting of the extraction unit 12, the prediction unit 14, and the determination unit 16 included in the controller 10 may include at least one of a software resource or a hardware resource. In the electronic device 1 in an embodiment, at least one selected from the group consisting of the extraction unit 12, the prediction unit 14, and the determination unit 16 may include specific means in which a software resource and a hardware resource operate in cooperation.

The extraction unit 12 extracts a feature of a heart rate of a subject from heart rate data acquired at the biological information acquisition unit 20. The prediction unit 14 estimates one or more internal states of the subject including, for example, a concentration level, a comprehension level, and engagement of the subject, while also predicting a change in biological information on the subject. The determination unit 16 determines whether the internal state of the subject estimated at the prediction unit 14 satisfies a predetermined condition. When the internal state of the subject satisfies the predetermined condition (for example, when the concentration level of the subject drops to or below a predetermined level), the determination unit 16 outputs a predetermined alert signal to the notification unit 40. The determination unit 16 also determines a difference between the biological information on the subject predicted at the prediction unit 14 and actual biological information. Note that the biological information acquisition unit 20 in the disclosure may be configured to acquire the heart rate data from the subject and extract a feature value of the heart rate.

In the disclosure, line-of-sight data extracted as data on a line of sight of a subject may serve as coordinate values (x, y) of a line-of-sight point. In the disclosure, the line-of-sight data may serve not only as the coordinates of the line-of-sight point of the subject but also as a feature value of the line of sight, such as a pupil diameter and/or rotation information on an eyeball. When the biological information on the subject is the heart rate, the feature value to be extracted may contain a chronological feature value of RR intervals calculated from the heart rate, a feature value of a frequency space, or a non-linear feature value, such as Poincaré Plot.

Further described blow is the operation of the controller 10 and the operation of the extraction unit 12, the prediction unit 14 and the determination unit 16 included in the controller 10.

The biological information acquisition unit 20 may include an image sensor, such as a digital camera, which electronically captures an image. The biological information acquisition unit 20 may include an imaging device performing photoelectric conversion, such as CCD (Charge Coupled Device Image Sensor) or CMOS (Complementary Metal Oxide Semiconductor) sensor. For example, the biological information acquisition unit 20 may supply a signal based on a captured image to the controller 10 or the like. To this end, the biological information acquisition unit 20 may be wiredly and/or wirelessly connected to the controller 10 as illustrated in FIG. 1. As long as the biological information acquisition unit 20 is able to capture an image of a subject, the biological information acquisition unit 20 is not limited to an imaging device, such as a digital camera, and may be any imaging device. For example, the biological information acquisition unit 20 with an infrared light camera used therefor may capture, as an image, a difference in a feature that reflects light, a difference in a feature that absorbs light, and/or the like. The biological information acquisition unit 20 may include an ECG (Electrocardiogram) sensor capable of acquiring the heart rate data. The biological information acquisition unit 20 may also include a non-contact sensor capable of acquiring the heart sound.

The biological information acquisition unit 20 captures an image of a subject. As described below, an assumed example of the subject may be a participant in an online conference. Specifically, in an embodiment, the biological information acquisition unit 20 captures, as the image of the subject, the image of the participant using a terminal connected to the online conference. In an embodiment, the biological information acquisition unit 20 may capture the image of the subject, for example, as a still image in every predetermined time period (e.g., every 30 frames per second). In an embodiment, the biological information acquisition unit 20 may capture the image of the subject, for example, as a continuously moving image. The biological information acquisition unit 20 may capture the image of the subject in one of a variety of data formats including RGB data and/or infrared data.

The biological information acquisition unit 20 may be configured, for example, as an in-camera of a terminal in order to capture the image of the participant of the online conference. The biological information acquisition unit 20 may be configured as a camera to be connected to a terminal. The controller 10 receives the image of the participant captured at the biological information acquisition unit 20. In the controller 10 as described below, the extraction unit 12 extracts the biological information including the line of sight of the participant from the image of the participant. To this end, the biological information acquisition unit 20 may be mounted at a location appropriate for capturing an image including the line of sight of the participant.

The storage unit 30 may have a function serving as a memory storing a variety of information. The storage unit 30 may store, for example, a program to be executed by the controller 10 and results of a process executed by the controller 10. The storage unit 30 may function as a working memory of the controller 10. To this end, the storage unit 30 may be wiredly and/or wirelessly connected to the controller 10 as illustrated in FIG. 1. The storage unit 30 may include, for example, at least one of a RAM (Random Access Memory) or a ROM (Read Only Memory). Although the storage unit 30 may include but be not limited to, for example, a semiconductor memory or the like and may be any storage device. For example, the storage unit 30 may be a storage medium, such as a memory card inserted into the electronic device 1 in an embodiment. The storage unit 30 may be an internal memory of a CPU used as the controller 10 or a different unit connected to the controller 10.

The storage unit 30 may store, for example, machine learning data. The machine learning data herein may be data generated through machine learning. The machine learning data may include a parameter generated through machine learning. Machine learning may be based on a technique of AI (Artificial Intelligence) that makes a specific task executable through training. More specifically, machine learning may include a technique of automatically constructing an algorithm or a model, in which an information processing apparatus, such as a computer, learns a large amount of data and executes a task of classification, prediction, and/or the like. In the description, at least part of the technique of AI may include a technique of machine learning. In the description, machine learning may include supervised learning that, based on correct answer data, learns a feature or a rule of input data. Machine learning may include unsupervised learning that learns the feature or the rule of the input data without the correct answer data. Machine learning may include reinforcement learning or the like that learns the feature or the rule of the input data by giving reward or punishment. In the description, machine learning may include any combination of supervised learning, unsupervised learning, and reinforcement learning.

Concept of machine learning data in the embodiment may include an algorithm that outputs a predetermined inference (estimation) result using an algorithm having learned the input data. In the embodiment, an algorithm outputting the predetermined inference (estimation) result may be one of a variety of algorithms including, for example, linear regression that predicts a relationship between an independent variable and a dependent variable, a neural network (NN) that mathematically models cranial nervous system neurons of human, a least-square method that performs calculation by squaring errors, a decision tree that represents problem solving in a tree structure, regularization that transforms data using a predetermined method, and the like. In the embodiment, a deep neural network may be used as one type of the neural network. The deep neural network is one type of neural network and generally signifies a network having a deep structure including one or more intermediate layers. Deep learning frequently serves as an algorithm configuring AI.

In an embodiment, information stored on the storage unit 30 may be information pre-stored, for example, before the time of shipment from a factory or information acquired as appropriate by the controller 10. In an embodiment, the storage unit 30 may store information received from a communication unit (a communication interface) connected to the controller 10, the electronic device 1, or the like. In such a case, the communication unit may receive a variety of information by wiredly or wirelessly communicating with, for example, an external electronic device, a base station, or the like. In an embodiment, the storage unit 30 may store information input to an input unit (an input interface) connected to the controller 10 or the electronic device 1. In such a case, a user of the electronic device 1 or another person may input a variety of information by operating the input unit.

In accordance with a predetermined signal (e.g., an alert signal) output from the controller 10, the notification unit 40 may output a predetermined alert drawing attention of the user of the electronic device 1. To this end, as illustrated in FIG. 1, the notification unit 40 may be wiredly and/or wirelessly connected to the controller 10. The notification unit 40 may be any functional unit that outputs, for example, sound, voice, light, characters, video, vibration, and/or the like as one or more predetermined alerts used to stimulate at least one selected from the group consisting of the sense of hearing, the sense of sight, and the sense of touch of the user. Specifically, the notification unit 40 may include at least one selected from the group consisting of, for example, an audio output unit, such as a buzzer or a speaker, a light emitting unit, such as an LED, a display, such as an LCD, and a tactile sensation presentation unit, such as a vibrator. In this way, the notification unit 40 may output the predetermined alert in response to the predetermined signal output from the controller 10. In an embodiment, the notification unit 40 may output the predetermined alert as information that acts on at least one selected from the group consisting of the sense of hearing, the sense of sight, and the sense of touch of a living thing, such as human.

In an embodiment, upon estimation that the concentration level of the subject, for example, as the internal state of the user, drops down to or lower than a predetermined threshold, the notification unit 40 may output an alert corresponding to a drop of the concentration level of the subject. In an embodiment, for example, the notification unit 40 outputting visual information may notify the subject themselves of the drop of the concentration level of the subject using light emission, a predetermined displaying, or the like. In an embodiment, the notification unit 40 outputting auditory information may notify the subject themselves of the drop of the concentration level of the subject using a predetermined sound, a predetermined voice, or the like. In an embodiment, the notification unit 40 outputting tactile information may notify the subject themselves of the drop of the concentration level of the subject using a predetermined vibration, or the like. In this way, the subject themselves may recognize the drop of the concentration level, for example, in an online conference.

Upon estimation that a concentration level of a participant in a conference as the subject has dropped down to or lower than a predetermined threshold, the notification unit 40 may notify either an organizer or a facilitator of the online conference, or another participant of the online conference of the presence of the participant whose concentration level has dropped. The notification unit 40 may notify in a variety of methods either the organizer or the facilitator of the online conference or another participant of the presence of the participant whose concentration level has dropped.

### (Estimation Operation Example of Internal State)

The following discussion relates to an example of an estimation operation of an internal state of a subject performed at the electronic device 1 in an embodiment.

The electronic device 1 estimates the internal state, such as the concentration level of a participant, by performing, with an autoencoder (auto encoder), machine learning on an image or the like of the subject as the participant of the online conference. The autoencoder is one of the architectures of neural network. The autoencoder may be a neural network including an encoder (hereinafter also referred to as a reference sign ENN) and a decoder (hereinafter also referred to as a reference sign DNN). In the electronic device 1 in an embodiment, the controller 10 may have a function serving as an autoencoder. Specifically, in an embodiment, the controller 10 in the electronic device 1 may have a function serving as the encoder ENN and the decoder DNN.

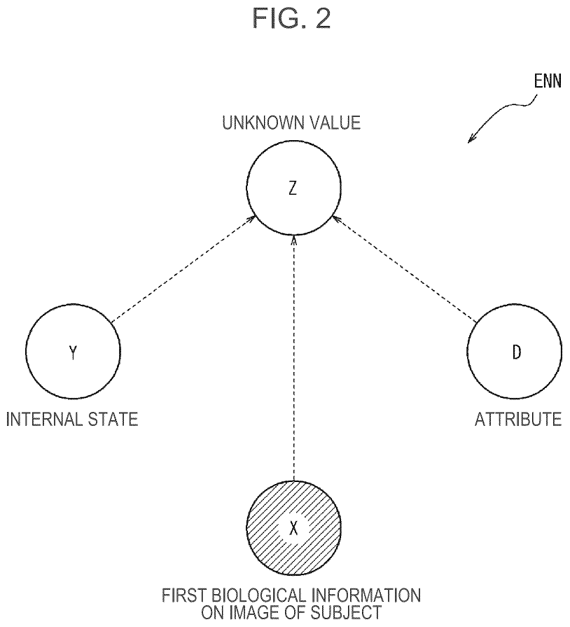

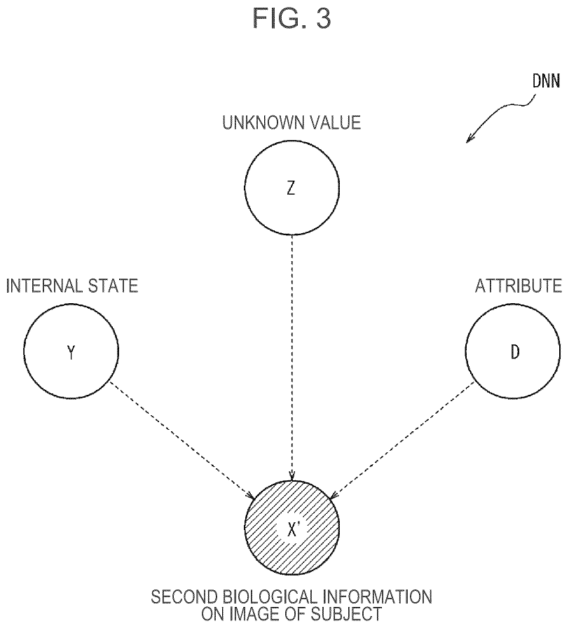

FIGS. 2 and 3 conceptually illustrate a neural network that functions as an autoencoder in the electronic device 1 in an embodiment. FIG. 2 conceptually illustrates the encoder. Specifically, FIG. 2 conceptually illustrates the encoder ENN in the neural network that functions as an autoencoder in the electronic device 1 in an embodiment. FIG. 3 conceptually illustrates a decoder and explains a principle that estimates the internal state, such as the concentration level, of the subject in accordance with an image of the subject (an operator) of the electronic device 1 in an embodiment. Specifically, FIG. 3 conceptually illustrates the decoder DNN of the neural network that functions as an autoencoder in the electronic device 1 in an embodiment.

As illustrated in FIG. 3, when the electronic device 1 in an embodiment estimates the internal state of the subject, a generation process assumed is that information Y indicating the internal state, an unknown value Z, and attribute information D cause second biological information X′ on the subject to be generated. The second biological information X′ herein may be information including an image of a line of sight of the subject (for example, an operator). The information Y indicating the internal state may be information indicating the internal state, such as the concentration level of the subject. The unknown value Z may include a latent variable that is unobservable. The attribute information D may include information representing at least one selected from the group consisting of, for example, sex, age, and age category (for example, twenties, thirties, or the like) of the subject. The attribute information D may include information indicating a role of the subject in the online conference. The attribute information D may include information representing a position of the subject (affiliation, position, or either authority or responsibility). The attribute information D may be pre-stored on the storage unit 30, acquired by causing the subject or the like to input the attribute information D as appropriate, or acquired from an external device or the like via communication as appropriate. In the disclosure, the information Y indicating the internal state may include, for example, a comprehension level, engagement, or the like of the subject in the online conference.

During machine learning of the electronic device 1 in an embodiment, as illustrated in FIG. 2, the electronic device 1 infers the unknown value Z from the first biological information X on the subject, the information Y indicating the internal state, and the attribute information D, using the encoder ENN of the neural network. The first biological information X on the subject herein may include an image of a line of sight of the subject (for example, a participant of the online conference). The image of the line of sight of the subject included in the first biological information X on the subject may be an image that the extraction unit 12 has extracted from the image of the subject captured at the biological information acquisition unit 20. The information Y indicating the internal state may include information indicating one or more internal states, including, for example, the concentration level, the comprehension level, and the engagement of the subject. The attribute information D may include information indicating an attribute, such as the age, the sex, and/or the like of the subject. The unknown value Z may include a latent variable that is unobservable. A phase of learning to estimate the internal state of the subject is hereinafter simply referred to as “learning phase”.

With the unknown value Z inferred as illustrated in FIG. 2, the use of the decoder DNN of the neural network illustrated in FIG. 3 may generate the second biological information X′ on the subject from the inferred unknown value Z, the information Y indicating the internal state, and the attribute information D. The second biological information X′ on the subject herein is information resulting from reconfiguring the first biological information X on the subject. The electronic device 1 in an embodiment may update a weight parameter of the neural network through error backpropagation while using as a loss function a degree of change of the second biological information X′ from the original first biological information X on the subject. The loss function may include a regularization term indicating a degree of deviation of a probability distribution of the unknown value Z from a predetermined probability distribution. The predetermined probability distribution may be, for example, a normal distribution. Kullback-Leibler divergence may be usable as a term representing the degree of deviation of the probability distribution of the unknown value Z from the predetermined probability distribution.

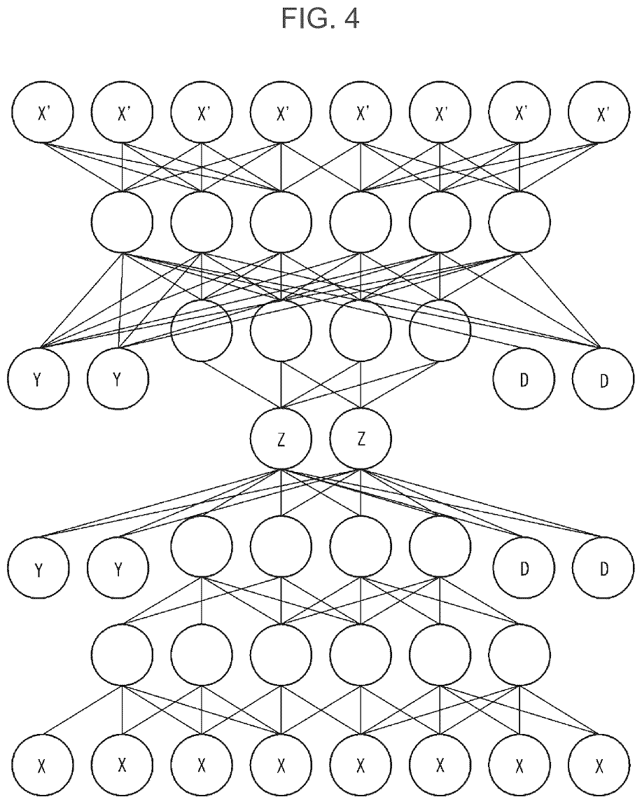

FIG. 4 conceptually illustrates implementation of the autoencoder in the electronic device 1 in an embodiment. An operation of the learning phase of the electronic device 1 in an embodiment is described first.

As illustrated in FIG. 4, in response to the supply of the first biological information X illustrated at the lowest row and further in response to the supply of the information Y indicating the internal state and the attribute information D, the electronic device 1 in an embodiment infers the unknown value Z illustrated at the intermediate row in FIG. 4. The electronic device 1 in an embodiment infers the unknown value Z, and then in response to the supply of the information Y indicating the internal state and the attribute information D, the electronic device 1 in an embodiment may acquire the second biological information X′ illustrated at the top row.

The autoencoder in the electronic device 1 in an embodiment may be configured to estimate the information Y indicating the internal state and the unknown value Z in response to the supply of only the first biological information X and the attribute information D. The attribute information D including information indicating the attribute, such as the age, the sex, and/or the like of the subject, may be pre-stored on the storage unit 30, input via the input unit, or received via the communication unit. The attribute information D may include information that the controller 10 or the like has estimated in accordance with the image of the subject captured at the biological information acquisition unit 20.

Referring to FIG. 4, in an embodiment, the autoencoder in the electronic device 1 reproduces the second biological information X′ on the subject, via the unknown value Z, from the first biological information X on the subject, the information Y indicating the internal state, and the attribute information D. Specifically, in the electronic device 1 in an embodiment, the autoencoder has a function that reconfigures at least one of the image of the line of sight of the subject or the feature value of the line of sight (the second biological information X′) in accordance with at least one of the image of the line of sight of the subject or the feature value of the line of sight (the first biological information X).

In the disclosure, the autoencoder may have a function that reconfigures at least one of the image of the line of sight of the subject or the feature value of the line of sight (the second biological information X′) in accordance with the biological information, such as the heart rate, other than the line of sight.

In the disclosure, at least one of the image of the line of sight of the subject or the feature value of the line of sight may include coordinate values (x, y) of the line-of-sight point. In the disclosure, the image of the line of sight of the subject and the feature value of the line of sight may include not only the coordinates of the-line-of-sight point but also the feature value of the line of sight, such as either a pupil diameter or rotation information on an eyeball, or a combination thereof. In the disclosure, extracting at least one of the image of the line of sight of the subject or the feature value of the line of sight is simply referred to as “extracting the line of sight”, “calculating the line of sight”, or the like. In the disclosure, estimating at least one of the image of the line of sight of the subject or the feature value of the line of sight is simply referred to as “estimating the line of sight”, “calculating the line of sight”, or the like. In the disclosure, at least one of the image of the line of sight of the subject or the feature value of the line of sight may include an image including an eyeball region. In the following discussion, since information input to the neural network is biological information, the information input to the neural network may be defined as line-of-sight information including the image including the eyeball region.

In the following discussion, the information input to the neural network may include biological information that is other than a line-of-sight image and includes the heart rate.

In an embodiment, for example, when the concentration level as the internal state is in a variety of states, the electronic device 1 may reconfigure the image of the line of sight of the subject or the feature value of the line of sight (the second biological information X′) to estimate the information Y indicating the internal state.

The controller 10 in the electronic device 1 may intentionally generate, for example, a state in which a participant of an online conference as a subject is fully concentrated on contents of the online conference. The controller 10 may intentionally generate, for example, a state in which a participant of an online conference as a subject is not fully concentrated on contents of the online conference.

The state in which the participant of the online conference is not fully concentrated on the contents of the online conference may be a state in which the attention of the participant is drawn to a task other than the online conference. For example, a generated state may be that during the online conference, the participant performs, as a task other than the online conference, work such as a predetermined mental calculation, unrelated to the online conference. Adjustment of complexity of the work may adjust the degree of state in which the participant fails to be fully concentrated on the contents of the online conference. For example, the complexity of the work may be adjusted in accordance with a difficulty level of a problem as a metal calculation. For example, a state in which the participant of the online conference performs a very simple mental calculation during the online conference may be determined to be a state in which the concentration level of the participant is relatively high while the participant is not fully concentrated on the contents of the online conference. A state in which the participant of the online conference performs a fairly complex mental calculation during the online conference may be determined to be a state in which the concentration level of the participant is relatively low.

Using the autoencoder in the electronic device 1 in an embodiment, the controller 10 may reconfigure the image of the line of sight of the subject or the feature value of the line of sight (the second biological information X′) corresponding to the information Y indicating the internal state of the participant of the online conference corresponding to each intentionally generated state.

As described above, when the internal state of the subject indicated by the information Y is in a variety of states, the electronic device 1 in an embodiment may reconfigure the image of the line of sight of the subject or the feature value of the line of sight (the second biological information X′). For example, the information Y indicating the internal state may be set to be Y=0 in a concentrated state and Y=1 in an unconcentrated state.

The controller 10 in the electronic device 1 may determine a validity of the information Y indicating the internal state in accordance with a degree of reproduction at which the image of the line of sight of the subject (the second biological information X′) reconfigured based on the information Y indicating the variety of internal states has reproduced an original image of the line of sight of the subject (the first biological information X). For example, if the degree of reproduction at which the image of the line of sight of the subject (the second biological information X′) reconfigured based on the information Y1 indicating a given internal state reproduces the original image of the line of sight of the subject or the feature value of the line of sight (the first biological information X) is high, the controller 10 may determine that the validity of information Y1 indicating the internal state is high (namely, close to the correct answer). On the other hand, if the degree of reproduction at which the image of the line of sight of the subject or the feature value of the line of the sight (the second biological information X′) reconfigured based on the information Y2 indicating the given internal state reproduces the original image of the line of sight of the subject or the feature value of the line of sight (the first biological information X) is low, the controller 10 may determine that the validity of information Y2 indicating the internal state is low (namely, far from the correct answer).

In this way, the electronic device 1 in an embodiment may adjust parameters of the encoder ENN and the decoder DNN in accordance with a reproducibility of the first biological information X through the second biological information X′. Further in addition to the reproducibility, the electronic device 1 may adjust the parameters of the encoder ENN and the decoder DNN in accordance with the loss function that includes a degree of distribution deviation that represents how far a probability distribution of the unknown value Z estimated at the encoder ENN deviates from a predetermined probability distribution. In this case, the predetermined distribution may be a normal distribution. Also, the case described above, the degree of distribution deviation may be Kullback-Leibler divergence.

### (Prediction Operation of Biological Information)

As described above, in an embodiment, the controller 10 in the electronic device 1 acquires, as an estimation result of the biological information, the second biological information X′ output from the autoencoder by inputting the first biological information X to the autoencoder described with reference to FIG. 4. Specifically, the controller 10 reconfigures the second biological information X′ in accordance with the first biological information X or the like.

### <Prediction Model 80 of Biological Information>

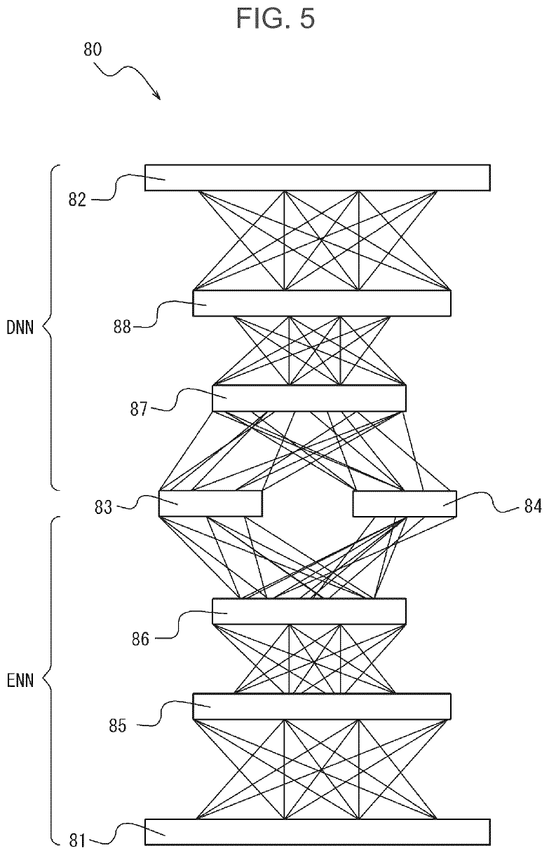

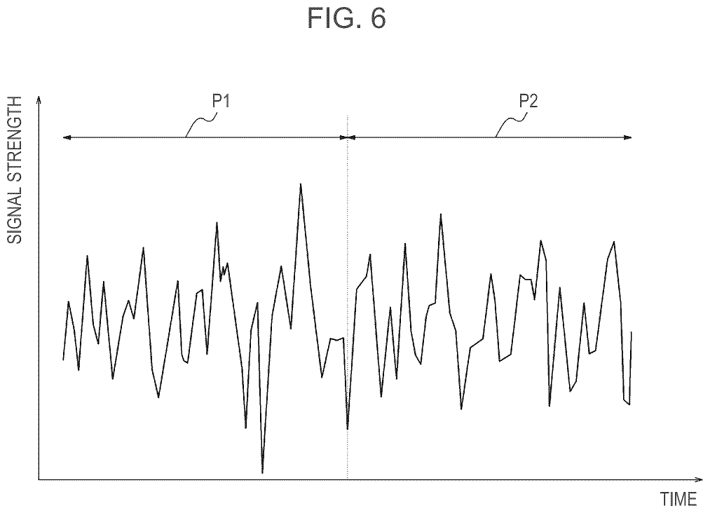

The internal state of the subject appears not only as the biological information at a certain time point but also a chronological change of the biological information. Thus, in order to acquire information on the chronological change of the biological information, the controller 10 may predict, as the second biological information X′, biological information in a period after a period of the acquisition of the first biological information X, using the prediction unit 14. In this case, the autoencoder may be configured to, when the biological information on the subject is input in a certain period, output a result of prediction of the biological information on the subject in a period after the certain period. The autoencoder outputting the prediction result of the biological information on the subject may implement a prediction model 80 illustrated in FIG. 5. The prediction model 80 is configured such that the prediction model 80 outputs the prediction result of biological information in a second period denoted by P2, upon receiving biological information in a first period denoted by P1 out of a signal waveform as a result of measuring the biological information on the subject as illustrated in FIG. 6. In a graph of the signal waveform in FIG. 6, the horizontal axis denotes time and the vertical axis denotes signal strength.

The prediction model 80 may include a plurality of layers configuring a neural network. The prediction model 80 include an input layer 81 and an output layer 82. When the input layer 81 receives the biological information in the first period, the prediction model 80 outputs a prediction result of the biological information in the second period from the output layer 82. The prediction model 80 may further include layers 83 and 84 representing unknown values. The prediction model 80 may further include intermediate layers 85 and 86 between the input layer 81 and the layers 83 and 84 representing the unknown values. The prediction model 80 may further include intermediate layers 87 and 88 between the layers 83 and 84 representing the unknown values and the output layer 82. The number of layers included in the prediction model 80 is not limited to seven layers illustrated in FIG. 5 but may be six layers or less or eight layers or more.

The prediction model 80 may estimate the unknown value Z on the subject in accordance with the biological information on the subject in the first period by processing information on each of the input layer 81 to the layers 83 and 84 representing the unknown values. The layers 83 and 84 representing the unknown values output an estimation result of the unknown value Z on the subject. A portion from the input layer 81 to the layers 83 and 84 representing the unknown values in the prediction model 80 corresponds to the encoder ENN. In the encoder ENN, the prediction model 80 is configured to receive the information Y indicating the internal state of the subject. In this case, the unknown value Z on the subject is estimated in accordance with the biological information on the subject in the first period and the information Y indicating the internal state. In the encoder ENN, the prediction model 80 may be configured to further receive the attribute information D on the subject. In this case, the unknown value Z on the subject is estimated in accordance with the biological information on the subject in the first period, the information Y indicating the internal state of the subject, and the attribute information D on the subject.

The prediction model 80 predicts the biological information on the subject in the second period in accordance with the estimation result of the unknown value Z on the subject by processing information on each layer from the layers 83 and 84 representing the unknown values to the output layer 82. The output layer 82 outputs the prediction result of the biological information on the subject in the second period. A portion from the layers 83 and 84 representing the unknown values to the output layer 82 in the prediction model 80 corresponds to the decoder DNN. In the decoder DNN, the prediction model 80 is configured to receive the information Y indicating the internal state of the subject. In this case, the biological information on the subject in the second period is estimated in accordance with the unknown value Z on the subject and the information Y indicating the internal state of the subject. In the decoder DNN, the prediction model 80 may be configured to further receive the attribute information D on the subject. In this case, the biological information on the subject in the second period is estimated in accordance with the unknown value Z on the subject, the information Y indicating the internal state of the subject, and the attribute information D on the subject.

The prediction model 80 may be configured as a learned model. The prediction model 80 may be generated by learning, as training data, the biological information in the first period and the biological information in the second period and the information Y indicating the internal state of the subject. The prediction model 80 may be generated by performing learning with the attribute information D on the subject further added to the training data.

The controller 10 may generate the prediction model 80. An example of a generation operation of the prediction model 80 is described below.

The controller 10 acquires the measurement result of the biological information in each of the first period and the second period. The controller 10 may acquire the biological information in a certain period and then segment the biological information in the first half and the second half of the certain period. The controller 10 may set the biological information in the first half to be the biological information in the first period and the biological information in the second half to be the biological information in the second period.

The controller 10 extracts the feature value of the biological information in each of the first period and the second period. When the biological information is the line of sight of the subject, the controller 10 may calculate, as the feature value of the line of sight, coordinates of a point which the subject looks at or an amount of movement of the coordinates. When the biological information is the heart rate of the subject, the controller 10 may calculate, as the feature value of the heart rate, a chronological feature value calculated from the heart rate, a feature value related to frequency, and/or a non-linear feature value.

The controller 10 inputs the measurement result of the biological information in the first period to the prediction model 80 and acquires the prediction result of the biological information in the second period from the prediction model 80. The controller 10 calculates a prediction error in accordance with the prediction result of the biological information in the second period and the actually acquired measurement result of the biological information in the second period. The controller 10 may calculate the prediction error in accordance with the feature value of the biological information. For example, when the biological information is the line of sight of the subject, the controller 10 may quantify, as the prediction error, a difference in the amount of movement of the coordinates of the line-of-sight point of the subject.

The controller 10 adjusts a parameter of each layer of the prediction model 80 such that the prediction error becomes smaller. The controller 10 may adjust the parameter of each layer of the prediction model 80 such that the prediction error becomes less than a determination threshold value. In order to predict the biological information in the second period in accordance with the biological information in the first period, the controller 10 may use the prediction model 80 in which the parameter is adjusted such that the prediction error is less than the determination threshold value.

The controller 10 may repeat the operation from the acquisition of the measurement result of the biological information to the calculation of the prediction error until the prediction error converges. When the prediction error becomes less than the determination threshold value, the controller 10 may determine that the prediction error has converged. In order to predict the biological information in the second period in accordance with the biological information in the first period, the controller 10 may use the prediction model 80 with the prediction error converged.

The controller 10 may further acquire the information Y indicating the internal state of the subject. The controller 10 may acquire the prediction result of the biological information in the second period by inputting the measurement result of the biological information in the first period to the prediction model 80 having received the information Y indicating the internal state of the subject and may calculate the prediction error. The controller 10 may adjust the parameter of each layer such that the prediction error becomes less than the determination threshold value in the prediction model 80 having received the information Y indicating the internal state of the subject.

The controller 10, for example, may intentionally generate the state in which the participant of the online conference as the subject is fully concentrated on only the contents of the online conference and may acquire the measurement result of the biological information in the first period and the second period in that state. The controller 10 may adjust the parameter of each layer such that the prediction error becomes less than the determination threshold value in the prediction model 80 that has received information indicating the state that the internal state of the subject is fully concentrated on the contents of the online conference.

The controller 10, for example, may intentionally generate the state in which the participant of the online conference as the subject is not fully concentrated on the contents of the online conference and may acquire the measurement result of the biological information in the first period and the second period in that state. The controller 10 may adjust the parameter of each layer such that the prediction error becomes less than the determination threshold value in the prediction model 80 that has received information indicating the state that the internal state of the subject is not fully concentrated on the contents of the online conference.

The information Y indicating the internal state of the subject may set to be, for example, Y=0 in the state that the subject is fully concentrated on only the contents of the online conference. The information Y indicating the internal state of the subject may set to be, for example, Y=1 in the state that the subject is not fully concentrated on the contents of the online conference. The information Y indicating the internal state of the subject may be set to any value from 0 to 1 depending on a degree of concentration at which the subject is concentrated on the contents of the online conference. The degree of concentration at which the subject is concentrated on the contents of the online conference is also referred to as a concentration level.

As described above, the controller 10 may adjust the parameters of the prediction model 80 such that the prediction error of the biological information in the second period becomes smaller. In other words, the controller 10 may adjust the parameters of the encoder ENN and the decoder DNN, in accordance with the reproducibility of the actual measurement result of the biological information in the second period that is based on the prediction result of the biological information in the second period responsive to the biological information in the first period. The controller 10 may adjust the parameters of the encoder ENN and the decoder DNN in accordance with not only the reproducibility but also the loss function including the degree of distribution deviation that indicates how far the probability distribution of the unknown value Z estimated at the encoder ENN deviates from the predetermined probability distribution. In this case, the predetermined probability distribution may be a normal distribution. Also, in the case described above, the degree of distribution deviation may be Kullback-Leibler divergence.

### <Estimation of Internal Information Based on Prediction of Biological Information>

The controller 10 acquires the prediction result of the biological information on the subject using the prediction model 80. The controller 10 estimates the internal state of the subject in accordance with the comparison of the prediction result of the biological information on the subject with the measurement result. An example of a specific estimation operation is described below.

The controller 10 assumes an internal state of the subject and generates information on the assumed internal state. The assumed internal state, in other words, information indicating a proforma internal state of the subject, is also referred to as proforma information. The controller 10 inputs to the encoder ENN and the decoder DNN in the prediction model 80 the proforma information in place of the information Y indicating the internal state of the subject.

The controller 10 inputs the measurement result of the biological information in the first period to the input layer 81 in the prediction model 80 having received the proforma information. The prediction model 80 having received the proforma information outputs from the output layer 82 the prediction result of the biological information in the second period on the subject when the internal state of the subject is an internal state indicated by the proforma information. The prediction result output from the prediction model 80 having received the proforma information is associated with the proforma information.

The controller 10 compares the measurement result of the biological information in the second period on the subject with the prediction result associated with the proforma information. As a difference between the measurement result and the prediction result is smaller, a difference between the internal state represented by the proforma information associated with the prediction result and the actual internal state is smaller. In other words, the controller 10 can determine the validity of the information Y indicating the internal state, in accordance with the degree of reproduction at which the biological information in the second period predicted based on the biological information in the first period and the information Y indicating the variety of internal states reproduces the actual biological information in the second period.

For example, the biological information in the second period predicted based on the information Y1 indicating a given internal state may reproduce the actual biological information in the second period at a high degree of reproduction. In this case, the controller 10 may determine that the validity of the information Y1 indicating the internal state is high (in other words, close to the correct answer).

On the other hand, an assumption may be that the biological information in the second period predicted in accordance with information Y2 indicating a certain internal state has reproduced the actual biological information in the second period only at a low degree. In such a case, the controller 10 may determine that the validity of the information Y2 indicating the internal state is low (in other words, far from the correct answer).

The controller 10 assumes a plurality of internal states as the internal state of the subject. The controller 10 inputs to the prediction model 80 the proforma information on each of the plurality of proforma internal states. The controller 10 inputs the measurement result of the biological information in the first period to the input layer 81 in the prediction model 80 having received the proforma information indicating each of the plurality of proforma internal states and acquires the prediction result of the biological information in the second period from the output layer 82. The prediction result acquired by the controller 10 is associated with each of a plurality of pieces of proforma information.

The controller 10 compares the measurement result of the biological information in the second period on the subject with the prediction result associated with each of the plurality of pieces of proforma information. The controller 10 may extract the prediction result having a difference from the measurement result falling within a predetermined range, and may estimate as the internal state of the subject the internal state indicated by the proforma information associated with the extracted prediction result. The controller 10 may decide a prediction result that has the smallest difference from the measurement result and may estimate as the internal state of the subject the internal state indicated by the proforma information associated with the decided prediction result.

When the controller 10 uses the concentration level of the subject as the information Y indicating the internal state of the subject, the controller 10 may assume the value of the concentration level to be multiple values. The value assumed to be the value of the concentration level is also referred to as a proforma value. The controller 10 may calculate a prediction error of the biological information in the second period in the prediction model 80 having received each of the multiple values. The controller 10 may estimate, to be the value of the concentration level of the subject, the proforma value that is input to the prediction model 80 with the prediction error less than the determination threshold value. The controller 10 may estimate, to be the value of the concentration level of the subject, the proforma value that is input to the prediction model 80 with the prediction error minimized.

The internal state of the subject may include an emotion of the subject. The emotion of the subject may include the concentration level of the subject. The emotion of the subject is not limited to the concentration level and may be indicated by joy/anger/grief/pleasure, a comprehension level, engagement with a conference, and/or the like. The emotion of the subject may be indicated by, for example, an index provided by a combination of a degree of joy, a degree of anger, a degree of grief, and a degree of pleasure. The emotion of the subject may be indicated by a sense of either comfort or discomfort or a sense of either security or anxiety, or the like. Information indicating the emotion of the subject is also referred to as an emotional label.

The controller 10 may generate the proforma information as a combination of multiple parameters, such as the concentration level, the emotion, or the like of the subject. The controller 10 may apply to the prediction model 80 the proforma information that is acquired by combining in a round-robin fashion all values that are assumed to be each parameter. The controller 10 may appropriately set the number of values that are assumed to be each of the parameters and may apply to the prediction model 80 the proforma information that is acquired by combining proforma values of the number that are set to be each of the parameters. The controller 10 may generate combinations of the values of each of the parameters in several narrowed patterns and may apply the proforma information on the generated combinations to the prediction model 80. The controller 10 may predict the biological information on the subject in each piece of the proforma information applied to the prediction model 80 and decide the proforma information that indicates an estimation result of the internal state of the subject in response to the prediction result.

The controller 10 may output an alert to the subject, the user, or the like in accordance with the estimation result of the internal state of the subject. When the estimation result of the internal state of the subject satisfies an alert condition, the controller 10 may output the alert. The alert condition may include, for example, the concentration level of the subject that is equal to or higher than the determination threshold value. The alert condition may include, for example, the value of the emotional label of the subject that is outside a determination range. For example, in response to the estimation of a drop in the concentration level of the subject, the controller 10 may determine that the alert condition is satisfied and the controller 10 may output the alert prompting the subject, the user, or the like to take a step to increase the concentration level of the subject. For example, when the emotion of the subject worsens, the controller 10 determines that the alert condition has been satisfied and may output the alert prompting the subject, the user, or the like to take a step to improve the emotion of the subject. The controller 10 may output the alert using the notification unit 40.

### <Operation Example of Prediction Model Generation Method>

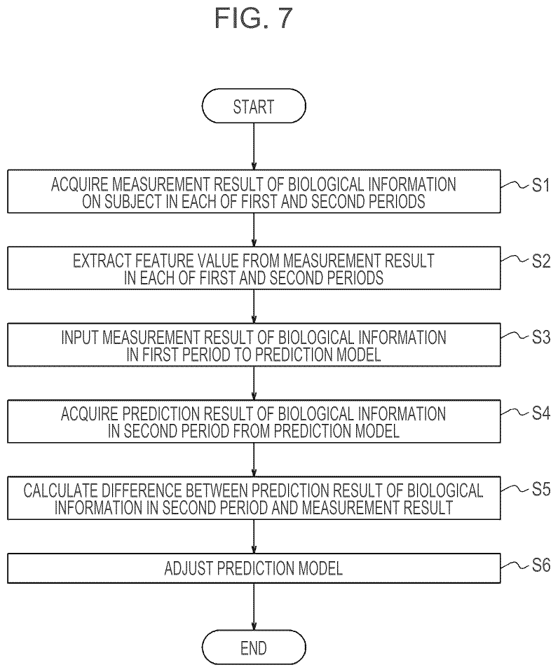

The controller 10 in the electronic device 1 may execute a method that generates a prediction model and includes an operation of a flowchart illustrated in FIG. 7. The method may be included in the control method of the electronic device 1. The method may be implemented as a program that generates the prediction model and is executed by a processor configuring the controller 10. The program may be stored on a non-transitory computer readable recording medium. The program may be included in the control program of the electronic device 1.

The controller 10 acquires the measurement result of the biological information on the subject in each of the first period and the second period (step S1). The controller 10 extracts the feature value from the measurement result of the biological information on the subject in each of the first period and the second period (step S2).

The controller 10 inputs the measurement result of the biological information in the first period to the prediction model 80 that is under learning (step S3). The controller 10 acquires the prediction result of the biological information in the second period from the prediction model 80 that is under learning (step S4).

The controller 10 calculates as the prediction error a difference between the prediction result of the biological information in the second period and the measurement result acquired in the operation in step S1 (step S5). The controller 10 adjusts in accordance with the prediction error the parameter of the prediction model 80 that is under learning (step S6).

After executing the operation in step S6, the controller 10 may end execution of a flowchart in FIG. 7 or may repeat learning of the prediction model 80 after returning to the operation in step S1. After executing the operation in step S6, the controller 10 may execute an operation to determine whether the prediction error becomes less than the determination threshold value. After executing the operation of step S6, the controller 10 may execute an operation to determine whether the prediction error has converged.

### <Operation Example of Method of Generating Prediction Model>

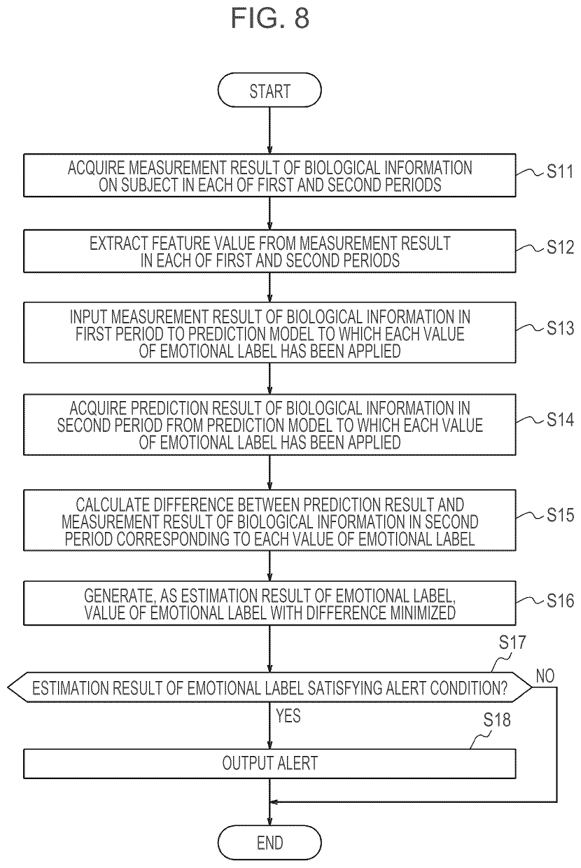

The controller 10 in the electronic device 1 may execute a method that estimates an internal state and includes operations in a flowchart illustrated in FIG. 8. The method may be included in the control method of the electronic device 1. The method may be implemented as a program that estimates the internal state and is executed by a processor configuring the controller 10. The program may be stored on a non-transitory computer readable recording medium. The program may be included in the control program of the electronic device 1.

The method illustrated in FIG. 8 estimates the emotional label as the information Y indicating the internal state of the subject.

The controller 10 acquires the measurement result of the biological information on the subject in each of the first period and the second period (step S11). The controller 10 extracts the feature value from the measurement result of the biological information on the subject in each of the first period and the second period (step S12).

The controller 10 applies to the prediction model 80 a proforma value of the emotional label as the proforma information on the internal state of the subject and inputs the measurement result of the biological information in the first period to the prediction model 80 to which each proforma value of the emotional label has been applied (step S13). The controller 10 acquires the prediction result of the biological information in the second period from the prediction model 80 to which each proforma value of the emotional label has been applied (step S14).

The controller 10 calculates a difference between the prediction result of the biological information in the second period and the measurement result of the biological information in the second period, corresponding to each proforma value of the emotional label (step S15). The controller 10 generates, as the estimation result of the emotional label, the value of the emotional label assumed when the difference is minimized (step S16).

The controller 10 determines whether the estimation result of the emotional label satisfies the alert condition (step S17). If the alert condition is not satisfied (step S17: NO), the controller 10 finishes the execution of the operations of the flowchart in FIG. 8. If the alert condition is satisfied (step S17: YES), the controller 10 outputs the alert using the notification unit 40 (step S18). After executing the operation in step S18, the controller 10 finishes the execution of the operations in the flowchart in FIG. 8.

### SUMMARY

As described above, the internal state of the subject may appear not only as the biological information at a given time point but also as a chronological change of the biological information. In the embodiment, the controller 10 in the electronic device 1 may predict the biological information on the subject and estimate the internal state of the subject in accordance with the prediction error. In this way, the controller 10 may estimate the internal state of the subject in view of the chronological change of the biological information. As a result, the controller 10 may improve the estimation accuracy of the internal state in comparison with the case that does not consider the chronological change of the biological information.

The controller 10 may estimate the internal state of the subject using multiple prediction models 80 that vary in lengths of the first period and the second period. The controller 10 may estimate the internal state of the subject using multiple prediction models 80 that vary in time from the end of the first period to the start of the second period. Considering a variety of patterns as the chronological change of the biological information may improve the estimation accuracy of the internal state. The controller 10 may estimate the internal state of the subject in view of the estimation result of the internal state that is obtained using the autoencoder reproducing the biological information in the first period. Considering the reproducibility of the biological information together with the chronological change of the biological information may improve the estimation accuracy of the internal state.

Generally, a line of sight, an attention-directing behavior, a heart rate, and/or the like of human may include large individual differences. For example, aged people have a narrower range of motion of the line of sight than young people. Therefore, in the estimation of the internal state of the subject, for example, appropriately considering the individual differences as described above may increase the estimation accuracy. Also, in the estimation of the internal state of the subject, a user may be easily convinced of the estimation result by allowing the user to be given an objective explanation about what model the estimation result is based on.

When the internal state, such as the concentration level of the subject, is estimated from an image captured from the subject, a comparative example is reverse in a causal relationship of the image and the internal state, in other words, assumes that learning is executed such that the internal state is estimated based on biological reaction data such as the line of sight or the like of the subject. Since the comparative example has a model structure reverse in the causal relationship, a data structure within the model has a black-box-like structure. Therefore, the comparative example may fail to identify a cause and may possibly lead to generating a model that has learned an erroneous structure. The causal relationship in the black-box structure causes objectively explaining the model of the causal relationship to the user to be difficult.

On the other hand, in the electronic device 1 in an embodiment, an algorithm estimating the internal state of the subject is based on a generative model different from a general recognition model or a general regression model. The generative model in the electronic device 1 learns from data a process that the internal state of the subject and the attribute of the subject (the age, the sex, or the like) cause the line of sight of the subject to be generated. For this reason, the electronic device 1 in an embodiment may improve the estimation accuracy considering the attribute of an individual as the subject. The electronic device 1 in an embodiment may objectively explain to the user a mechanism reflecting a data generation process. In an embodiment, the electronic device 1 may rationally estimate the internal state, such as the concentration level of the subject, in accordance with the data generation process.

### Alternative Embodiments

Alternative embodiments are described below.

### <Certainty Factor of Alternative Embodiments>

The prediction model 80 may be configured to output as a prediction result of biological information a result represented by a probability distribution. The probability distribution may be, for example, a normal distribution or the like. The prediction result of the biological information, if represented by a normal distribution, may be represented by a mean value and a standard deviation.

The controller 10 may compare the prediction result of the biological information represented as a probability distribution with an actual measurement result. The controller 10 may calculate as the prediction error a difference between the mean value of the probability distribution of the prediction result of the biological information and the actual measurement result of the biological information. The controller 10 may estimate as the internal state of the subject an internal state indicated by the proforma information that has been applied to the prediction model 80 having acquired the prediction error less than the determination threshold value. The controller 10 may estimate as the internal state of the subject an internal state indicated by the proforma information that has been applied to the prediction model 80 having acquired a minimum prediction error.

The controller 10 may calculate an index used to verify certainty (plausibility) of the estimation result of the internal state of the subject. The index verifying the certainty (plausibility) of the estimation result of the internal state of the subject is also referred to as the certainty factor.

In accordance with the probability distribution of the prediction result of the biological information, the controller 10 may calculate, for example, a probability at which an actual measurement result appears and may regard the calculated probability as the certainty factor of the internal state of the subject. In this case, as the estimation result of the internal state is more certain, the value of the certainty factor becomes larger. The controller 10 may output the estimation result of the internal state if the certainty factor is equal to or larger than the determination threshold value or may re-estimate the internal state if the certainty factor is smaller than the determination threshold value.

The controller 10 may regard, as the estimation result of the internal state of the subject, for example, the following equation (1) that uses mean value μ of the prediction result of the biological information, actual measurement result x, and standard deviation σ of the prediction result of the biological information.

$$[ Eq . 1 ]$$

$$\frac{1}{\sqrt{2 π} σ} exp ( - \frac{{( x - μ )}^{2}}{2 {σ}^{2}} ) \qquad ( 1 )$$

Specifically, the controller 10 may square a value that is obtained by dividing, by the standard deviation of the prediction result of the biological information, a difference between the mean value of the prediction result of the biological information and the actual measurement result and may regard, as the certainty factor of the internal state of the subject, the equation of the normal distribution expressed by using e. In this case, as the estimation result of the internal state is more certain, the certainty factor becomes larger.

The certainty factor is not limited to the equation described above but may be represented by 1/σ or 1/σ². The controller 10 may output the estimation result of the internal state if the certainty factor is smaller than the determination threshold value or may re-estimate the internal state if the certainty factor is equal to or larger than the determination threshold value.

As described above, the controller 10 calculates the certainty factor of the estimation result of the internal state of the subject and may determine in accordance with the certainty factor whether to output the estimation result of the internal state of the subject or to re-estimate the internal state of the subject. In this way, the estimation accuracy may be increased.

### <Operation Example of Method of Estimating Biological Information in View of Certainty Factor>

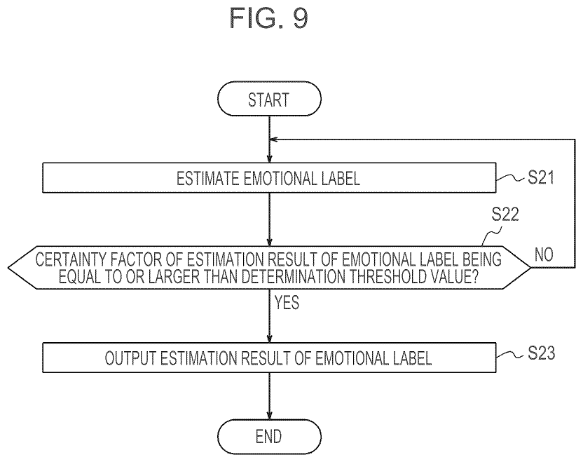

The controller 10 in the electronic device 1 may execute a method that generates a prediction model and includes operations in a flowchart illustrated in FIG. 9. The method may be included in the control method of the electronic device 1. The method may be implemented as a program that generates the prediction model and is executed by a processor configuring the controller 10. The program may be stored on a non-transitory computer readable recording medium. The program may be included in the control program of the electronic device 1.

Note that the method illustrated in FIG. 9 uses the emotional label as the information Y indicating the internal state of the subject.

The controller 10 estimates the emotional label of the subject by executing operations in steps from S11 to S16 in the flowchart illustrated in FIG. 8 (step S21). The controller 10 calculates the certainty factor of the estimation result of the emotional label and determines whether the certainty factor is equal to or larger than the determination threshold value (certainty factor threshold value) (step S22). If the certainty factor is neither equal to or nor larger than the determination threshold value (step S22: NO), in other words, the certainty factor is smaller than the determination threshold value, the controller 10 re-estimates the emotional label of the subject after returning to the operation in step S21. If the certainty factor is equal to or larger than the determination threshold value (step S22: YES), the controller 10 outputs the estimation result of the emotional label (step S23). The controller 10 finishes the execution of the operations in the flowchart in FIG. 9 after the execution of the operation in step S23.

### <Example of Executing Entity Estimating Internal State>

As described above, in an embodiment of the disclosure, the electronic device 1 may be configured as a terminal used to participate in an online conference. Also, the terminal may execute the method of an embodiment of the disclosure to generate the prediction model or the method of an embodiment of the disclosure to estimate the internal state.

Herein, the online conference may be conducted using a server apparatus or a cloud system, connected via a network or the like. In an embodiment of the disclosure, the electronic device 1 may be configured as a server apparatus or a cloud system that provides the online conference to the terminal. Also, in an embodiment of the disclosure, the server apparatus or the cloud system that provides the online conference to the terminal may execute the method of an embodiment of the disclosure to generate the prediction model or the method of an embodiment of the disclosure to estimate the internal state.

In other words, in an embodiment of the disclosure, the electronic device 1 may be configured to be an apparatus that serves as a host for the online conference. The host apparatus that serves as the host for the online conference may execute the method of an embodiment of the disclosure to generate the prediction model or the method of an embodiment of the disclosure to estimate the internal state.

The server apparatus, the cloud system or the host apparatus, each providing the online conference, may output to the terminal the estimation result of the internal state or may output to the terminal an alert responsive to the estimation result of the internal state.

Any person skilled in the art may perform a variety of modifications and changes in the disclosure. Therefore, these modifications and changes fall within the scope of the disclosure. For example, in each of the embodiments, a functional unit, means, or a step may be added in another embodiment in a logically consistent manner or may respectively replace a functional unit, means, or a step in another embodiment. In each of the embodiments, a plurality of functional units, a plurality of means, or a plurality of steps may be unified respectively into one functional unit, one means, or one step, or may be respectively segmented. Each of the embodiments of the disclosure described above is not limited to a faithful implementation of a respective embodiment but may be implemented by appropriately combining features of the embodiments or deleting a subset of the features.

In an embodiment of the disclosure, the electronic device 1 may be mounted on, for example, a mobile object. The mobile object may include, for example, a vehicle, a ship, or an aircraft. In an embodiment of the disclosure, when the electronic device 1 is mounted on the mobile object, the electronic device 1 may estimate a specific internal state (for example, a specific state of mind, emotion, a concentration level or the like) of a person (a driver or a passenger) on board the mobile object, such as a passenger car. The electronic device 1 may estimate as the internal state of a driver driving the mobile object the internal state of the driver, such as the emotion, the concentration level, or the like of the driver during driving. The electronic device 1 may be configured as a server apparatus or a cloud system, communicably connected to a terminal mounted on the mobile object.

The vehicle may include, for example, an automobile, an industrial vehicle, a railway vehicle, a vehicle for daily life, or a fixed-wing aircraft running on a runway. The automobile may include, for example, a passenger car, a truck, a bus, a two-wheeled vehicle, or a trolley bus. The industrial vehicle may include, for example, an industrial vehicle for agriculture or construction. The industrial vehicle may include, for example, a forklift or a golf cart. The industrial vehicle for agriculture may include, for example, a tractor, a cultivator, a transplanting machine, a binder, a combine harvester, or a lawn mower. The industrial vehicle for construction may include, for example, a bulldozer, a scraper, an excavator, a crane truck, a dump truck, or a road roller. The vehicle may include one that is run by human power. The category of the vehicle is not to be limited to the examples described above. The automobile may include, for example, an industrial vehicle capable of running on a road. The same vehicle may belong to multiple categories. The ship may include, for example, a watercraft (personal watercraft (PWC)), a boat, or a tanker. The aircraft may include, for example, a fixed-wing aircraft or a rotary-wing aircraft. The “user” and the “subject” may be a person who drives the mobile object, such as the vehicle or the aircraft, or may be a passenger who does not drive the mobile object.

### REFERENCE SIGNS

1 electronic device (10: controller, 12: extraction unit, 14: prediction unit, 16: determination unit, 18: line-of-sight prediction unit, 20: biological information acquisition unit, 21: first biological information acquisition unit, 22: second biological information acquisition unit, 30: storage unit, 40: notification unit)
80 prediction model (81: input layer, 82: output layer, 83 and 84: layers indicating unknown values, 85, 86, 87, and 88: intermediate layers)
ENN encoder
DNN decoder

1. An electronic device comprising a controller configured to estimate an internal state of a subject in accordance with a measurement result of biological information on the subject in each of a first period and a second period later than the first period,
wherein the controller is configured to
generate proforma information corresponding to a proforma internal state of the subject in the first period;
predict the biological information on the subject in the second period in accordance with the proforma internal state of the subject and the measurement result of the biological information on the subject in the first period; and
estimate an internal state corresponding to the biological information in accordance with a difference between the predicted biological information and the measurement result of the biological information on the subject in the second period.

2. The electronic device according to claim 1, wherein the controller is configured to calculate a certainty factor of an estimation result of the internal state of the subject, and determine, in accordance with the certainty factor, whether the estimation result of the internal state of the subject is output or estimation of the internal state of the subject is remade.

3. The electronic device according to claim 1, wherein the controller is configured to estimate the internal state of the subject further in accordance with attribute information on the subject.

4. A control method for an electronic device estimating an internal state of a subject in accordance with a measurement result of biological information on the subject in each of a first period and a second period later than the first period, the control method comprising:
at the electronic device, generating proforma information indicating each of a plurality of states assuming the internal state of the subject;
at the electronic device, generating proforma information corresponding to a proforma internal state of the subject in the first period;
at the electronic device, predicting the biological information on the subject in the second period, in accordance with the proforma internal state of the subject and the measurement result of the biological information on the subject in the first period; and
at the electronic device, estimating an internal state corresponding to the biological information, in accordance with a difference between the predicted biological information and the measurement result of the biological information on the subject in the second period.

5. The control method for the electronic device according to claim 4, further comprising:
at the electronic device, calculating a certainty factor of an estimation result of the internal state of the subject; and
at the electronic device, determining, in accordance with the certainty factor, whether the estimation result of the internal state of the subject is output or estimation of the internal state of the subject is remade.

6. The control method for the electronic device according to claim 4, wherein the electronic device estimates the internal state of the subject further in accordance with attribute information on the subject.

7. A non-transitory computer-readable medium storing a control program for an electronic device estimating an internal state of a subject in accordance with a measurement result of biological information on the subject in each of a first period and a second period later than the first period, the control program comprising:
causing the electronic device to generate proforma information indicating each of a plurality of states assuming the internal state of the subject;
causing the electronic device to generate proforma information corresponding to a proforma internal state of the subject in the first period;
causing the electronic device to predict the biological information on the subject in the second period in accordance with the proforma internal state of the subject and the measurement result of the biological information on the subject in the first period; and
causing the electronic device to estimate an internal state corresponding to the biological information in accordance with a difference between the predicted biological information and the measurement result of the biological information on the subject in the second period.

8. The non-transitory computer-readable medium according to claim 7, wherein the control program for the electronic device further comprises:
causing the electronic device to calculate a certainty factor of an estimation result of the internal state of the subject; and
causing the electronic device to determine, in accordance with the certainty factor, whether the estimation result of the internal state of the subject is output or estimation of the internal state of the subject is remade.

9. The non-transitory computer-readable medium according to claim 7, wherein the control program causes the electronic device to estimate the internal state of the subject further in accordance with attribute information on the subject.
